(19) United States
(12) Patent Application Publication
(10) Pub. No.: US 2024/0261137 A1
(43) Pub. Date: Aug. 8, 2024

(54) INSERTER SYSTEM FOR ROD IMPLANTS AND METHODS OF USE

(71) Applicant: Genentech, Inc., South San Francisco, CA (US)

(72) Inventors: Joshua David HORVATH, San Ramon, CA (US); Anna Lucia VOGEL, San Francisco, CA (US); Rachel Paige ARNOTT, San Francisco, CA (US); Ariel Ephraim WAITZ, Minnetonka, MN (US); Russell Richard HENNING, Hollister, CA (US); Bruce Rodney DENDULK, Ripon, CA (US); Laurence Edward TRIGG, Santa Clara, CA (US)

(21) Appl. No.: 18/407,200

(22) Filed: Jan. 8, 2024

Related U.S. Application Data
(63) Continuation of application No. PCT/US2022/036295, filed on Jul. 7, 2022.
(60) Provisional application No. 63/203,082, filed on Jul. 7, 2021.

(51) Int. Cl. A61F 9/00

(52) U.S. Cl. CPC A61F 9/0017; A61F 2230/0019

(57) ABSTRACT

Provided herein are devices and methods for delivering medical implants into a patient, such as intravitreally. In one exemplary implementation, an implant delivery device includes an elongated body, cannula, plunger, dispense button and prime button. The cannula is sized and configured to slidably receive a rod-shaped implant. The plunger is slidably received within the cannula. The delivery device is configured to be operated by a user moving the prime button from a locked position to an unlocked position, and subsequently moving a dispense button from an extended position to a depressed position, thereby causing the plunger to move from a retracted position to a distally extended position to eject the implant from a distal portion of the cannula. The device may further include an interlock mechanism that prevents the cap from moving from an attached position to a detached position when the prime button is in the locked position.

### CROSS REFERENCE TO RELATED APPLICATIONS

This application is a continuation of International Application No. PCT/US2022/036295, filed Jul. 7, 2022, which claims the benefit of U.S. Provisional Application No. 63/203,082, filed Jul. 7, 2021, each of which is herein incorporated by reference in its entirety for all purposes.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

The present disclosure relates to drug delivery systems and methods that can be used to introduce implants containing therapeutic or active agents, including bioerodible implants, into various locations within a patient, such as the eye, including into the vitreous of the eye.

A primary difficulty in treating diseases of the eye is the inability to introduce drugs or therapeutic agents into the eye and maintain these drugs or agents at a therapeutically effective concentration in the eye for the necessary duration. Systemic administration may not be an ideal solution because unacceptably high levels of systemic dosing are often needed to achieve effective intraocular concentrations, thus increasing the incidence of unacceptable side effects of the drugs. Simple ocular instillation or application is not an acceptable alternative in many cases because the drug may be quickly washed out by tear-action or may otherwise be depleted from the eye into the general circulation. Available methods make it difficult to maintain therapeutic levels of drug for adequate time periods.

Efforts to address this problem have led to the development of drug delivery devices, or implants, which can be implanted into the eye such that a controlled amount of desired drug can be released constantly over a period of several days, weeks, or even months. Many such devices have been previously reported. See, for example, U.S. Pat. No. 4,853,224, which discloses biocompatible implants for introduction into an anterior segment or a posterior segment of an eye for the treatment of an ocular condition. In addition, U.S. Pat. No. 5,164,188 discloses a method of treating an ocular condition by introduction of a biodegradable implant comprising drugs of interest into the suprachoroidal space or pars plana of the eye. See also U.S. Pat. Nos. 5,824,072; 5,476,511; 4,997,652; 4,959,217; 4,668,506; and 4,144,317. Other methods include anchoring a plug or tack containing a drug into the sclera of the eye (see, e.g., U.S. Pat. No. 5,466,233).

Various sites exist in the eye for implantation of a drug delivery device or implant, such as the vitreous of the eye, anterior or posterior chambers of the eye, or other areas of the eye including intraretinal, subretinal, intrachoroidal, suprachoroidal, intrascleral, episcleral, subconjunctival, intracorneal or epicorneal spaces. Wherever the desired location of implantation, typical methods of implantation all require relatively invasive surgical procedures, pose a risk of excessive trauma to the eye, and require excessive handling of the implant. For example, in a typical method for placement in the vitreous, an incision is made through the sclera, and the implant is inserted into and deposited at the desired location in the vitreous, using forceps or other like manual grasping device. Once deposited, the forceps (or grasping device) are removed, and the incision is sutured closed. Alternatively, an incision can be made through the sclera, a trocar can be advanced through the incision and then the implant can be delivered through the trocar. Similar methods can be employed to deliver implants to other locations, e.g., implantation in the anterior chamber of the eye through an incision in the cornea.

There are numerous drawbacks of such techniques for implant delivery. Extensive handling of the implant is necessitated in these techniques, creating a risk that the implant will be damaged in the process. Many implants are polymer-based and are relatively fragile. If portions of the implants are damaged and broken-off, the effective therapeutic dose delivered by the implant once placed will be significantly altered. In addition, it becomes inherently difficult using these methods to achieve reproducible placement from patient to patient. Additionally, all of these techniques require an incision or puncture in the eye large enough to require suturing. Thus, such techniques are typically performed in a surgical setting.

Many considerations affect the design and efficacy of an implant delivery device. First, it is important to ensure that the implant is consistently delivered to the subject with each application. Second, because implant therapy often requires numerous applications, the cost of providing the implant should also be considered.

Based on the foregoing, a need for a more facile, convenient, less invasive, and less traumatic means for delivering implants into the eye and other anatomy remains. In addition, a need for a more controlled means of delivering implants also remains. The innovations described herein solve these unmet needs and provide additional advantages.

### SUMMARY OF THE DISCLOSURE

According to aspects of the present disclosure, an implant delivery device may be provided with an elongated body, a cannula, a plunger, a dispense button and a prime button. In some embodiments, the elongated body has a proximal end and a distal end. The cannula is carried by the body and has a distal portion extending from the distal end of the body. The cannula is sized and configured to slidably receive a rod-shaped implant therein. The plunger is slidably received within the cannula and movable from a retracted position, in which a rod-shaped implant is retained inside the cannula, to a distally advanced position, in which the implant is ejected from the cannula by the plunger. The dispense button is movable from an extended position, in which the plunger remains in the retracted position, to a depressed position, in which the plunger is moved toward the distally advanced position. The prime button is movable from a locked position, in which the dispense button is prevented from moving into the depressed position, to an unlocked position, in which the dispense button may be allowed to move toward the depressed position. The delivery device is configured to be operated by a user moving the prime button from the locked position to the unlocked position, and subsequently moving the dispense button from the extended position to the depressed position, thereby causing the plunger to move from the retracted position to the distally extended position to eject the implant from the distal portion of the cannula.

In some embodiments, the prime button is located on the proximal end of the elongated body and moves towards the distal end of the body when moving from the locked position to the unlocked position. The device may be configured so that the prime button can be operated by a user's thumb. In some embodiments, the dispense button is located on a top side surface of the elongated body and generally moves radially inward when moving from the extended position to the depressed position. The device may be configured so that the dispense button can be operated by a user's forefinger or thumb.

In some embodiments, the device further comprises a cap movable from an attached position, in which the cap is covering the distal end of the body and the distal portion of the cannula, to a detached position, in which the cap is separated from the body. The device may further comprise an interlock mechanism that prevents the cap from moving from the attached position to the detached position when the prime button is in the locked position. In some embodiments, the device further comprises an interlock mechanism that prevents the dispense button from moving to the depressed position when the cap is in the attached position.

In some embodiments, the device further comprises a status window through a portion of the body. The status window allows a user to see a position of a movable part within the body which represents a priming status and an implant status, the movable part being movable between a locked position, an unlocked position and a dispensed position. The position of the movable part may be depicted by a change of color. In some embodiments, the change of color comprises a red feature showing through the status window when the movable part is in the locked position, a yellow feature showing through the status window when the movable part is in the unlocked position, and a green feature showing through the status window when the movable part is in the dispensed position. The position of the movable part may be depicted by a change of letter or symbol. In some embodiments, the change of letter or symbol comprises an L showing through the status window when the movable part is in the locked position, a U showing through the status window when the movable part is in the unlocked position, and a D showing through the status window when the movable part is in the dispensed position.

In some embodiments, the device further comprises a spring configured to return the prime button to the locked position if the prime button is not fully moved to the unlocked position. In these embodiments, the device is configured to provide tactile confirmation when the prime button is moved to the unlocked position, thereby confirming an unlocked state. The device may be configured to emit a soft click when the prime button reaches the unlocked position.

In some embodiments, the device further comprises a spring configured to return the dispense button to the extended position if the dispense button is not fully moved to the depressed position. In these embodiments, the device is configured to provide tactile confirmation when the dispense button is moved to the depressed position, thereby confirming an implant has been dispensed. The device may be configured to emit a soft click when the dispense button reaches the depressed position.

In some embodiments, the cannula is provided with a retaining feature configured to releasably retain a rod-shaped implant within the cannula until the implant is pushed out by the plunger. The retaining feature may comprise a bend in the cannula having an angle of at least 5 degrees. In some embodiments, the retaining feature comprises one or more items in a group consisting of a bend, a nail head, a dimple, an oval, a flex section, an adhesive, an S-curve, an etched sleeve and a wire. The device may further comprise a preloaded rod-shaped implant. In some embodiments, the device further comprises a window through the elongated body allowing a user to view the preloaded implant before it is dispensed by the device.

In some embodiments, the device further comprises a push bar coupled to the plunger and configured to move longitudinally within the elongated body. In these embodiments, the push bar is configured to be moved distally by the prime button between a locked position and an unlocked position. The push bar is configured to be moved further distally by the dispense button between the unlocked position and a dispensed position to drive the plunger distally within the cannula and dispense an implant from the distal portion of the cannula. The push bar may be provided with cam features to unlock a cap positioned on the distal end of the body when the push bar reaches the unlocked position. In some embodiments, the device is configured to dispense the rod-shaped implant intravitreally.

In some embodiments, a device for delivering a rod-shaped implant is provided with an elongated body, a cannula, a plunger, a dispense button, a cap, a prime button, and a status window. In these embodiments, the elongated body has a proximal end and a distal end. The cannula is carried by the body and has a distal portion extending from the distal end of the body. The cannula is sized and configured to slidably receive a rod-shaped implant therein. The plunger is slidably received within the cannula and is movable from a retracted position, in which a rod-shaped implant is retained inside the cannula, to a distally advanced position, in which the implant is ejected from the cannula by the plunger. The dispense button is located on a top side surface of the elongated body and is generally movable radially inward from an extended position, in which the plunger remains in the retracted position, to a depressed position, in which the plunger is moved toward the distally advanced position. The cap is movable from an attached position, in which the cap is covering the distal end of the body and the distal portion of the cannula and prevents the dispense button from moving into the depressed position, to a detached position, in which the cap is separated from the body and allows the dispense button to move into the depressed position. The prime button is located on the proximal end of the elongated body and is movable towards the distal end of the body from a locked position, in which the cap is prevented from moving into the detached position, to an unlocked position, in which the cap is allowed to move into the detached position. The status window is configured to indicate when the prime button has moved from the locked position to the unlocked position, and when the dispense button has moved into the dispense position. The delivery device is configured to be operated by a user moving the prime button from the locked position to the unlocked position, subsequently moving the cap from the attached position to the detached position, and subsequently moving the dispense button from the extended position to the depressed position, thereby causing the plunger to move from the retracted position to the distally extended position to eject the implant from the distal portion of the cannula.

According to aspects of the present disclosure, a method of delivering a rod-shaped implant includes the step of providing an implant delivery device. In this method, the implant delivery device includes an elongated body, a cannula, a plunger, a dispense button, a cap and a prime button. The elongated body has a proximal end and a distal end. The cannula is carried by the body and has a distal portion extending from the distal end of the body. The cannula carries a rod-shaped implant therein and the plunger is slidably received within the cannula. The dispense button is located on the elongated body. The cap is removably attached to the distal end of the body and covers the distal portion of the cannula when the cap is attached to the body. The prime button is located on the proximal end of the elongated body. The method further includes the step of moving the prime button from a locked position to an unlocked position, thereby unlocking the cap. After unlocking the cap by moving the prime button, the cap is removed from the body, thereby unlocking the dispense button. The method also includes the step of inserting a distal tip of the cannula into a patient. After unlocking the dispense button by removing the cap, the dispense button is moved from an extended position to a depressed position, thereby causing the plunger to move distally to eject the implant from the distal portion of the cannula and into the patient.

In some embodiments, the step of moving the prime button comprises placing a thumb on the button and moving the button in a distal direction. Moving the dispense button may comprise placing a forefinger on the button and moving the button in a radially inward direction. In some embodiments, the rod-shaped implant is dispensed intravitreally.

### BRIEF DESCRIPTION OF THE DRAWINGS

The novel features of the disclosure are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1 is a perspective view of an exemplary embodiment of an implant delivery device constructed and operated according to aspects of the present disclosure;

FIG. 2 is an exploded view of the device shown in FIG. 1;

FIG. 3 is an exploded view of the device cap and dispense button shown in FIG. 1;

FIG. 4 is a partially exploded view of the internal mechanism and left side enclosure of the device shown in FIG. 1;

FIG. 5 is an exploded view of the internal mechanism shown in FIG. 4;

FIG. 6 is a side cross-sectional view of the device shown in FIG. 1;

FIGS. 7A-7J are side cross-sectional views of implant retaining features;

FIGS. 8A-8H are side cross-sectional views of needle stop features;

FIG. 9 is a perspective view of an advancement mechanism of a second exemplary embodiment of an implant delivery device constructed and operated according to aspects of the present disclosure;

FIG. 10 is a side view of the advancement mechanism of FIG. 9;

FIGS. 11A-11D are a series of side views of the advancement mechanism of FIG. 9 and illustrating an exemplary sequence of operation;

FIG. 12 is a perspective view showing a prototype device utilizing the advancement mechanism of FIG. 9;

FIG. 13 is an exploded plan view showing components of the device of FIG. 12;

FIG. 14 is an exploded perspective view showing components of the device of FIG. 12;

FIG. 15 is a perspective view of a third exemplary embodiment of an implant delivery device constructed and operated according to aspects of the present disclosure;

FIG. 16 is an exploded plan view showing components of the device of FIG. 15;

FIG. 17 is a perspective view showing an exemplary rack and pinion mechanism of the device of FIG. 15;

FIG. 18 is an exploded perspective view showing components of the device of FIG. 15;

FIG. 19 is a perspective view of a fourth exemplary embodiment of an implant delivery device constructed and operated according to aspects of the present disclosure;

FIG. 20A is a perspective view of an exemplary advancement mechanism of the device of FIG. 19;

FIG. 20B is a side view showing internal and external components of the device of FIG. 19;

FIG. 20C is a side view of the exemplary advancement mechanism of FIG. 20A;

FIGS. 21A-21D are a series of side views of the advancement mechanism of FIG. 20A and illustrating an exemplary sequence of operation;

FIG. 22 is a perspective view showing internal components of the device of FIG. 19; and

FIG. 23 is an exploded perspective view showing components of the device of FIG. 19.

### DETAILED DESCRIPTION

Described herein are exemplary devices and methods for delivering rod-shaped implants subcutaneously, intravitreally, intramuscularly, intraarticularly, intravascularly or into other anatomy. In some embodiments, an implant dispense button and a device cap are both locked in place until a prime button on a proximal end of the device is “clicked” like a ball-point pen. Once primed, the cap is unlocked but the dispense button remains locked until after the cap is removed. Various features are provided that indicate to a medical practitioner whether the device has been fully primed, and whether the implant has been fully dispensed from the device into the patient.

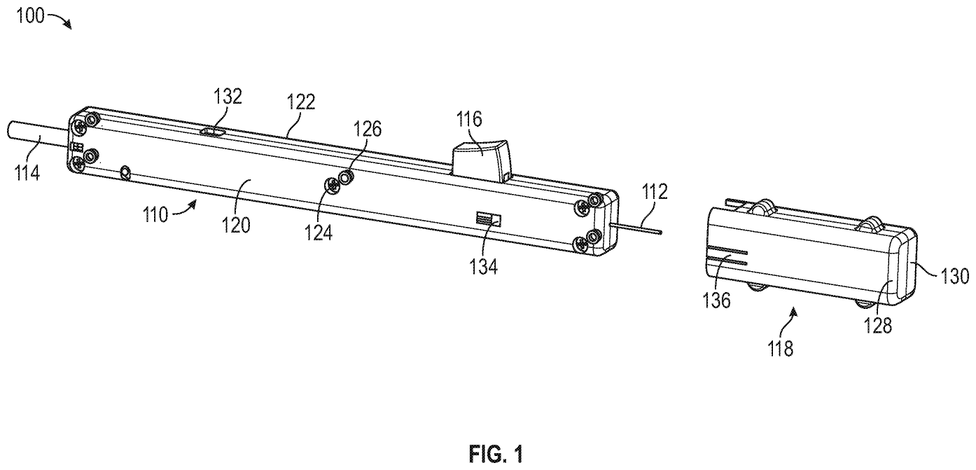

Referring to FIG. 1, an exemplary embodiment of an implant delivery device 100 is provided. Device 100 includes an elongated enclosure 110 having an implant dispensing needle 112 extending from its distal end and a prime button 114 extending from its proximal end. In this exemplary embodiment, a dispense button 116 is located atop enclosure 110 such that it can be depressed by a user's forefinger or thumb to dispense a rod-shaped implant from cannula 112. A cap 118 may be configured to be slidably received over the distal end of enclosure 110 to cover needle 112 when device 100 is not in use.

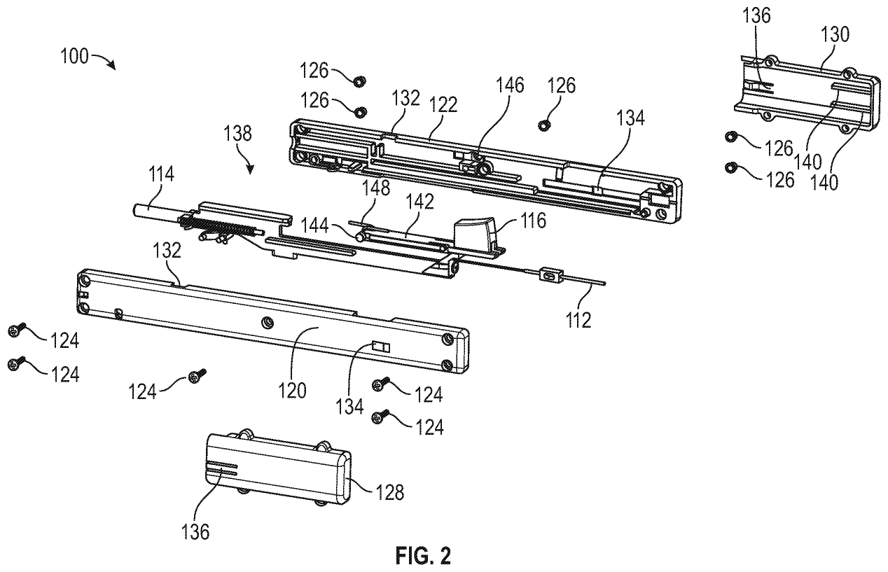

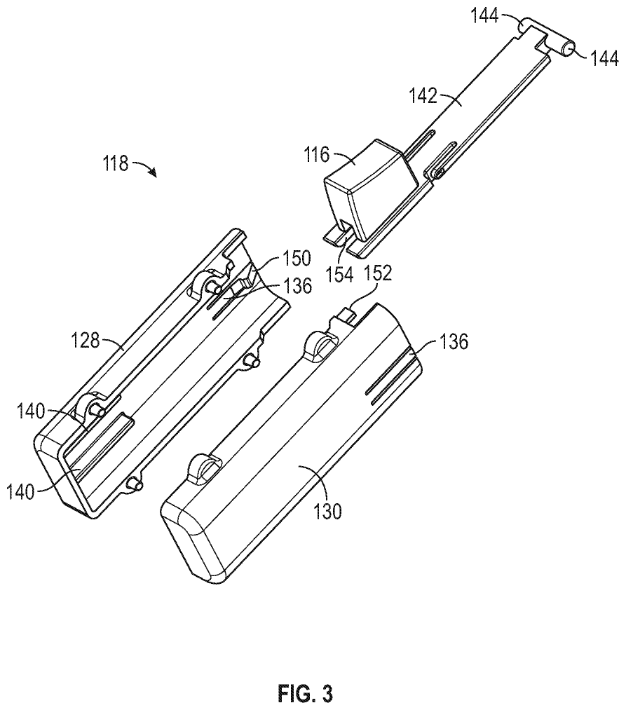

In this exemplary embodiment, enclosure 110 is formed from a right side 120 and a left side 122. The two sides of enclosure 110 are held together with five fasteners 124 which pass through the enclosure and engage threaded inserts 126. Cap 118 is also be formed from a right side 128 and a left side 130. The two sides of cap 118 may be held together with four pins formed on right side 128 that engage with four recesses on left side 130 with a snap or wedge fit (as best seen in FIGS. 2 and 3.)

The top of enclosure 110 may be provided with a status indicator window 132, as will be subsequently described in more detail. Each side of the distal end of enclosure 110 may be provided with recesses 134, one of which is shown in FIG. 1. Recesses 134 are configured to releasably receive resilient arms 136 of cap 118. Arms 136 cooperate with recesses 134 to lock cap 118 in place over the distal end of enclosure 110 until the cap is unlocked by prime button 114, as will be subsequently described in more detail.

Referring to FIG. 2, a partially exploded view of implant delivery device 100 is provided. Internal mechanism 138 (sometimes referred to as an interlock mechanism) with its various moving parts resides between prime button 114 and needle 112. When device 100 is assembled, internal mechanism 138 is sandwiched between the right side 120 and left side 122 of the enclosure, which are held together by five fasteners 124 coupled to five threaded inserts 126, as previously mentioned. The right side 128 and left side 130 of the device cap are also shown. Both sides of the cap may be provided with stops 140 which abut against the distal end of enclosure sides 120 and 122 when the cap is being placed onto the enclosure. This arrangement provides sufficient space within the cap to ensure that needle 112 is protected by the cap but is not contacted by it.

In this exemplary embodiment, implant dispense button 116 is mounted or formed on the distal end of a dispense button lever 142. The proximal end of lever 142 is provided with a pair of laterally protruding pins 144. The right side 120 and left side 122 of the enclosure each include a pivot recess 146 on their inner surface for receiving one of the pins 144 of lever 142. When the device is assembled, lever 142 allows dispense button 116 to move in a generally downward direction into the enclosure. Torsion spring 148 may be provided to bias dispense button 116 in a generally upward direction out of the enclosure.

Referring to FIG. 3, an exploded view of device cap 118 along with implant dispense button 116 is provided. As can be seen on the right side 128 of cap 118, the free end of each resilient arm 136 is provided with an inwardly protruding barb 150. Barbs 150 include a ramped surface which faces away from cap 118 as shown. These ramped surfaces allow the cap to easily slide onto the distal end of the device. Resilient arms 136 flex outwardly as cap 118 is being attached to the device. Barbs 150 also include a barbed surface facing into the cap, as shown. When the cap is fully installed on the device, resilient arms 136 urge barbs 150 into recesses 134 (shown in FIGS. 1 and 2) and these barbed surfaces catch on the distal edges of recesses 134. This arrangement retains cap 118 on the device until it is unlocked, as will be subsequently described in more detail.

Cap 118 may include a longitudinally extending locking tab 152. When cap 118 is installed on the implant dispensing device, locking tab 152 extends into a recess 154 in dispense button 116. Locking tab 152 prevents implant dispensing button 116 from being depressed until after cap 118 is removed. With this arrangement, the implant cannot be inadvertently ejected from the device while the cap is on.

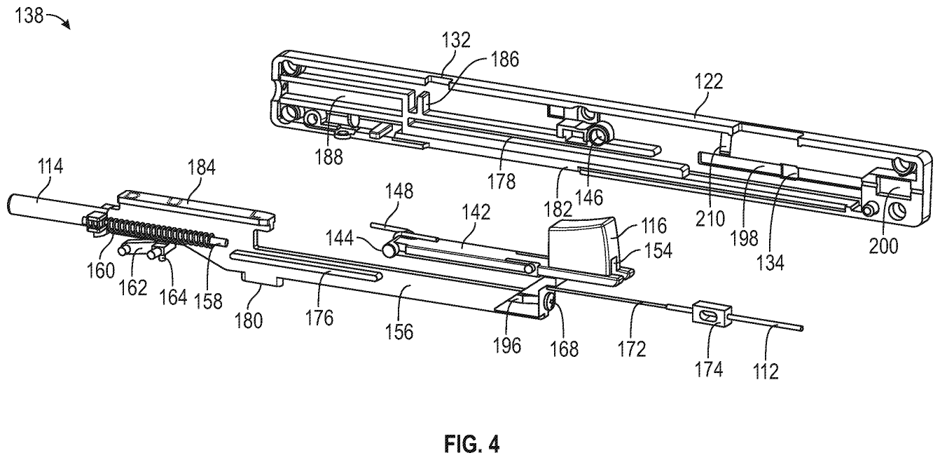

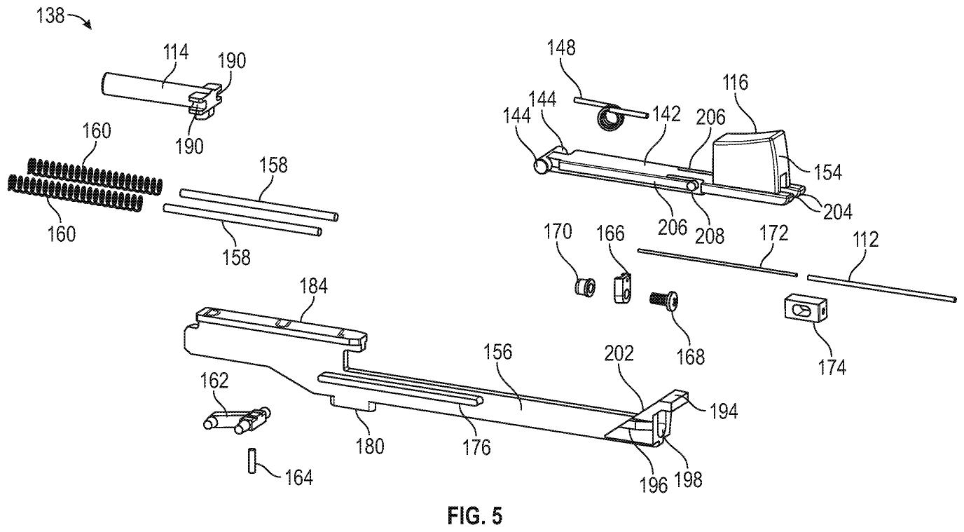

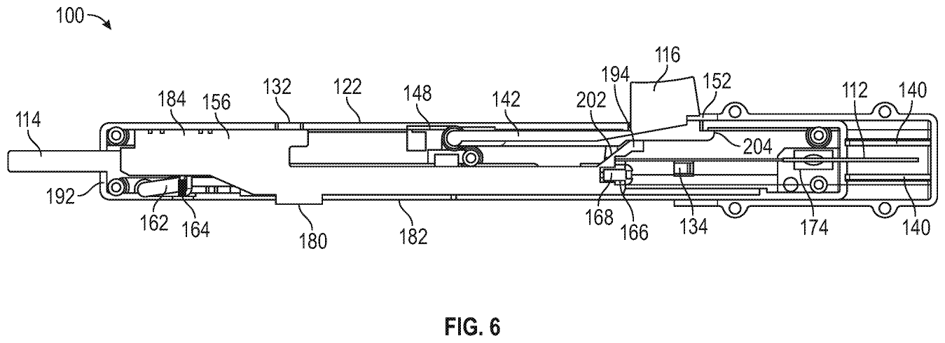

Referring to FIGS. 4-6, the construction and operation of internal mechanism 138 will be described. FIG. 4 is an enlarged view of the internal mechanism 138 and left side 122 of the enclosure shown in FIG. 2, FIG. 5 is an exploded view of internal mechanism 138, and FIG. 6 is a cross-sectional side view of device 100. Internal mechanism 138 includes implant dispense button 116, button lever 142, torsion spring 148, pusher 156, a pair of dowels 158, a pair of compression springs 160 received over dowels 158, prime button 114, prime button latch 162, compression spring 164 coupled to latch 162, plunger retainer 166, fastener 168, threaded insert 170, plunger rod 172, needle 112 and needle mount 174.

In this exemplary embodiment, a main component of internal mechanism 138 is pusher 156, sometimes referred to as a push bar. Pusher 156 includes laterally protruding ribs 176 that are slidably received in slots 178 of both sides of the enclosure (one slot 178 shown in left enclosure 122 in FIG. 4.) Pusher 156 also includes a downwardly protruding rib 180 that is slidably received in slot 182 formed between the bottom edges of the enclosure. This arrangement allows pusher 156 to longitudinally slide within the enclosure between three distinct positions: 1) a proximal-most Locked position (as shown in FIG. 6); 2) an intermediate Unlocked position; and 3) a distal-most Dispensed position. The letters L, U and D may be formed on a top flange surface 184 of pusher 156 such that they each show through status indicator window 132 (shown in FIGS. 4 and 6) in succession to indicate the state of the device to the user. In other embodiments, different colors, symbols and or other letters or features may be used to show the device status. For example, the position of pusher 156 and the state of the device may be depicted by a change of color, wherein the change of color comprises a red feature showing through window 132 when pusher 156 is in the Locked position, a yellow feature showing through window 132 when pusher 156 is in the Unlocked position, and a green feature showing through window 132 when pusher 156 is in the Dispensed position.

Prime button 114 is used to drive pusher 156 distally from the Locked position to the Unlocked position, and implant dispense button 116 is used to drive pusher 156 further distally from the Unlocked position to the Dispensed position.

As best depicted in FIG. 4, each dowel 158 is fixedly located in one of the enclosure sides when the device is assembled (not shown), extending to dowel stop 186. Each spring 160 is slidably located over dowel 158 in spring box 188. As shown in FIG. 5, the distal end of prime button 114 is provided with two laterally protruding guide channels 190. Each guide channel 190 is configured to slide along one of the dowels 158 and compress one of the springs 160 as prime button 114 is depressed distally.

As best seen in FIG. 6, the distal end of prime button 114 abuts the proximal end of pusher 156 to drive it distally. Prime button latch 162 is pivotably mounted between the enclosure halves such that it can pivot about a pair of opposing pins formed near its proximal end (best seen in FIG. 5.) Compression spring 164 urges the distal end of latch 162 upwardly against the bottom of pusher 156. As prime button 114 is depressed distally against the return force of springs 160, a downwardly extending ramped surface on the bottom of its distal end rides along the top surface of latch 162, forcing the distal end of latch 162 to pivot downwardly against spring 164. As prime button 114 reaches the Unlocked position, the distal end of latch 162 pivots upwardly into engagement with locking surface 192, thereby latching prime button 114 and pusher 156 in the Unlocked position and preventing them from returning proximally. If the device user releases prime button 114 before it is fully depressed, compression springs 160 return the button to its fully extended position, signaling to the user that the device has not been unlocked. However, when the user fully depresses prime button 114, the above-described arrangement provides an audible and or tactile click, signaling to the device user that the device has been unlocked. In some embodiments, the click is a “soft click”, meaning that it is strong enough to be heard and or felt by the device user, but not so strong that it would cause inadvertent tool movement that would then cause the patient to move.

As best seen in FIG. 5, the distal end of pusher 156 includes an upwardly extending locking surface 194. As shown in FIG. 6, when pusher 156 is in the proximal-most Locked position, locking surface 194 prevents dispense button 116 from being downwardly depressed. When pusher 156 is moved distally into the Unlocked position, locking surface 194 clears the distal end of button lever 142, thereby unlocking button 116. However, if the cap remains on the device, it prevents button 116 from being depressed, as previously described in reference to FIG. 3.

As best seen in FIG. 4, the distal end of pusher 156 includes a pair of laterally protruding tabs 196 (only one tab 196 shown in FIG. 4.) Tabs 196 each have a ramped leading edge and slide in slots 198 on the interior surfaces of the enclosure sides (only one slot 198 shown in FIG. 4.) When pusher 156 reaches the Unlocked position, tabs 196 are adjacent to recesses 134 in the enclosure sides. In this position, tabs 196 urge barbs 150 on the ends of resilient arms 136 of cap 118 (shown in FIG. 3) outward such that they clear recesses 134 and allow the cap to be removed from the device.

As best seen in FIG. 5, the distal end of pusher 156 includes a recess configured to receive plunger retainer 166. When the device is assembled, plunger 172 is rigidly affixed in a hole through retainer 166. Threaded insert 170 is rigidly affixed in recess 198, allowing retainer 166 to be attached to pusher 156 with fastener 168. With this arrangement, plunger 172 is rigidly affixed to pusher 156, extends distally therefrom, and moves longitudinally therewith within the device. When the device is assembled, needle 112 is rigidly affixed in a hole through needle mount 174, as shown in FIG. 4. Needle mount 174 in turn resides in opposing pockets 200 formed in the left side 122 (shown) and right side (not shown) of the enclosure. This arrangement holds needle 112 in a fixed position relative to the enclosure.

When the internal mechanism is assembled, as shown in FIG. 4, the distal end of plunger 172 is slidably received within the proximal end of needle 112. As pusher 156 is moved distally from the Locked position to the Unlocked position, and then to the Dispenses position, plunger 172 extends progressively further through needle 112. In the Locked position, plunger 172 extends into about 15% of the length of needle 112. A rod-shaped implant (not shown) may be preloaded into the central lumen of the needle, located distally from the distal end of plunger 172. When pusher 156 is moved to the Unlocked position, plunger 172 occupies about 50% of the length of needle 112. When pusher 156 is moved to the Dispensed position, plunger 172 pushes the rod-shaped implant out of needle 112 and into the patient. In this position, the distal tip of plunger 172 may extend slightly from the distal end of needle 112 to ensure that the implant is fully clear from the needle.

As previously described, prime button 114 is used to drive pusher 156 from the distal-most Locked position to the intermediate Unlocked position. Once pusher 156 reaches the

Unlocked position and device cap 118 is removed, dispense button 116 is unlocked and can be depressed by the user to drive pusher 156 from the Unlocked position to the Dispensed position, in which the implant is delivered into the patient. As best seen in FIGS. 5 and 6, the distal end of pusher 156 may be provided with an upwardly and proximally facing ramped surface 202, and the distal end of dispense button lever 142 may be provided with mating downwardly and distally facing ramped surfaces 204. When pusher 156 is in the Unlocked position, its ramped surface 202 is situated directly below ramped surface 204 associated with dispense button 116. As dispense button 116 is pressed inwardly (downwardly) against the return force of torsion spring 148, ramped surface 204 slides down along ramped surface 202 and urges pusher 156 distally toward the Dispensed position.

As best seen in FIG. 5, a pair of resilient arms 206 may be provided on opposite sides of dispense button arm 142. An outwardly protruding dimple 208 may be provided on the distal tip of each arm 142. As best seen in FIG. 4, left side 122 of the enclosure (as well as the right side, not shown) may be provided with a curved slot 210 with an inwardly protruding dimple located at the bottom. As dispense button 116 is depressed, dimples 208 travel along slot 210. If the device user releases button 116 before it is fully depressed, torsion spring 148 returns the button to its fully extended position, signaling to the user that the implant has not been fully dispensed. However, when the user fully depresses dispense button 116, dimples 208 travel past the dimples at the bottom of slots 210. Resilient arms 206 momentarily flex inward as the dimples pass each other, and then flex back. This arrangement provides an audible and or tactile click and locks button 116 in the depressed position, signaling to the user that the implant has been fully dispensed. In some embodiments, the click is a “soft click”, meaning that it is strong enough to be heard and or felt by the device user, but not so strong that it would cause inadvertent tool movement that would then cause the patient to move.

Referring again to FIG. 1, an exemplary method of operation of device 100 will be summarized. Device 100 may be provided with a pre-loaded rod-shaped implant, may be pre-sterilized and provided in a sealed container. The device is removed from the sterile container and remains in a locked state. The letter L is displayed in status indicator window 132 to indicate that device 100 is in the locked state. In this locked state, implant dispense button 116 is locked from being depressed, both externally (as previously described in reference to FIG. 3) and internally (as previously described in reference to FIG. 6.) Additionally, cap 118 cannot be removed from main enclosure 110 before device 100 is unlocked.

After the patient has been prepared to receive the implant, a surgeon or other medical practitioner presses prime button 114 to unlock the device. An audible and or tactile click is provided when prime button 114 is fully depressed, and button 114 becomes locked in the fully depressed position. These are all cues to the medical practitioner that device 100 has been unlocked. Additionally, the letter U is displayed in status indicator window 132 to indicate that device 100 is in the unlocked state.

Once device 100 is in the unlocked state, cap 118 may be removed from main enclosure 110. At this point, implant dispense button 116 is fully unlocked. The medical practitioner then inserts needle 112 into the patient, such as subcutaneously, intravitreally, intramuscularly, intraarticularly, intravascularly or into other anatomy. When the distal tip of needle 112 is located in the implant target location, the medical practitioner presses button 116 to dispense the implant from needle 112. An audible and or tactile click is provided when dispense button 116 is fully depressed, and button 116 becomes locked in the fully depressed position. These are all cues to the medical practitioner that the implant has been dispensed. Additionally, the letter D is displayed in status indicator window 132 to indicate that the implant has been dispensed.

After the implant has been dispensed from device 100 into the patient, device 100 may be moved proximally until needle 112 is fully withdrawn from the patient. In some embodiments, device 100 is a single use device. Cap 118 may be replaced over needle 112 and or device 100 may be discarded.

In some embodiments, the length of enclosure 110 is no more than about 156 mm, the height is no more than about 16 mm and the width is no less than about 8 mm. In other embodiments (not shown), internal mechanism 138 may slimmed down and or reconfigured so that it fits into a generally cylindrical enclosure, such as that of a large pen. In some of these embodiments, the diameter of the enclosure is at least about 8 mm and no larger than about 16 mm. In some embodiments, the diameter is about 11.4 mm. In some embodiments, prime button 114 extends at least 19.5 mm from enclosure 110 when in its extended state. In some embodiments, the distal face of dispense button 116 is located 30 mm from the distal end of enclosure 110 and extends no higher than about 12.7 mm above the enclosure.

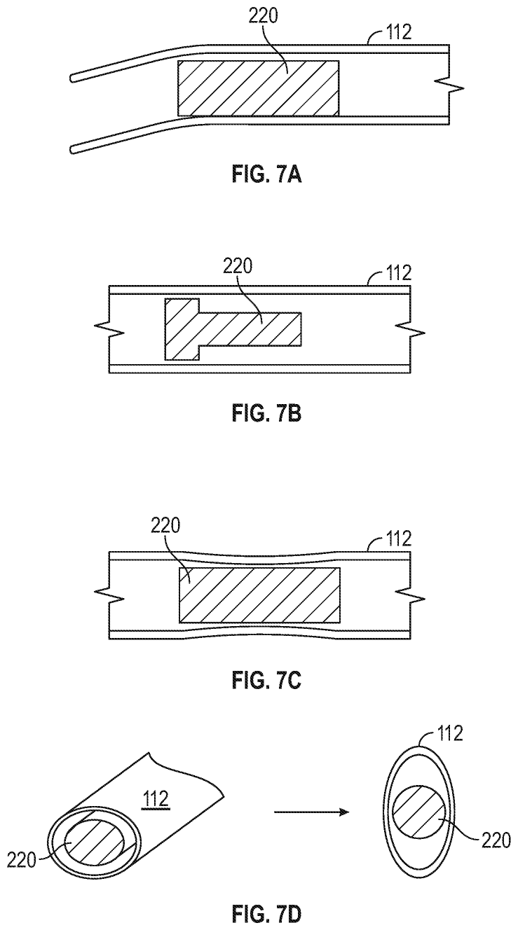

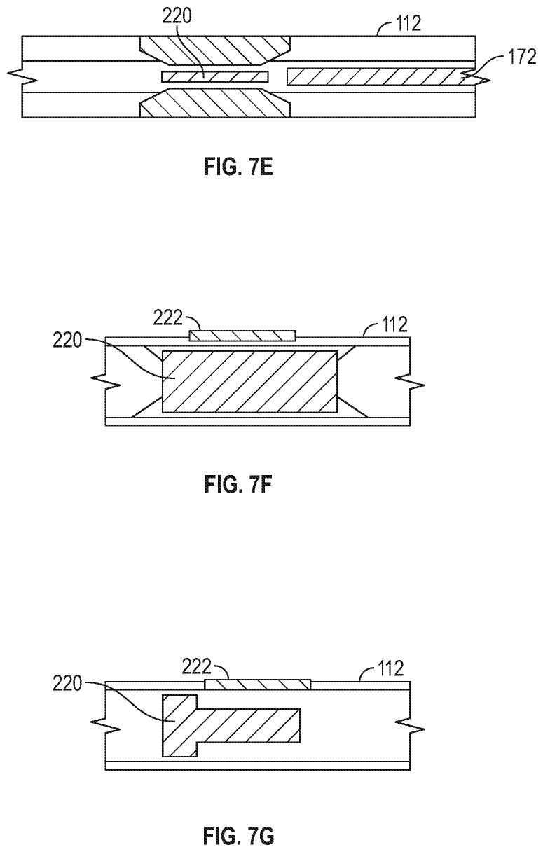

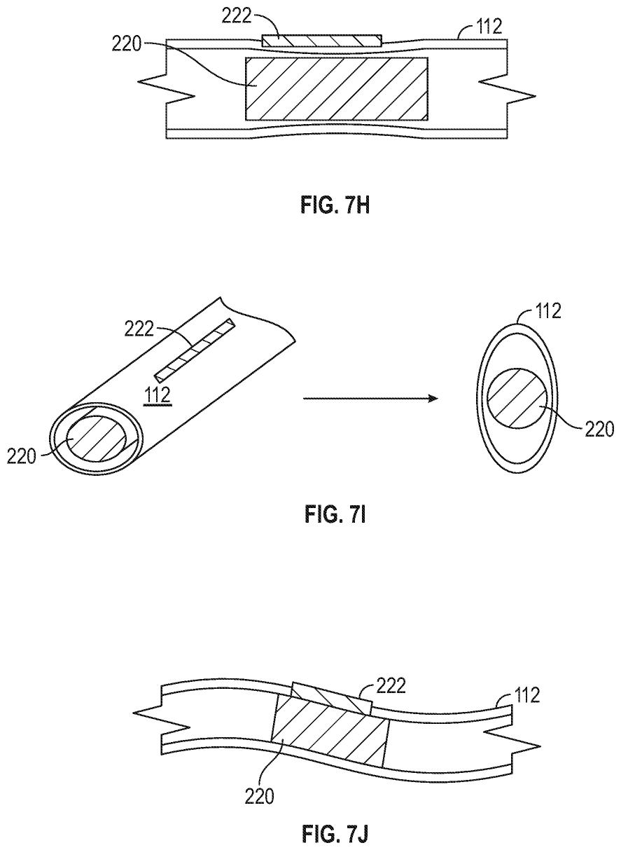

Referring to FIGS. 7A-7J, one or more features may be added to needle 112 and or the rod-shaped implant 220 to ensure the implant remains in needle 112 until it is pushed out by plunger 172, yet does not stick in the needle or adversely affect the actuation force. A window 222 may be provided as shown in FIGS. 7F-7J to permit the user to see the pre-loaded implant 220 and confirm its location and or condition before it is implanted.

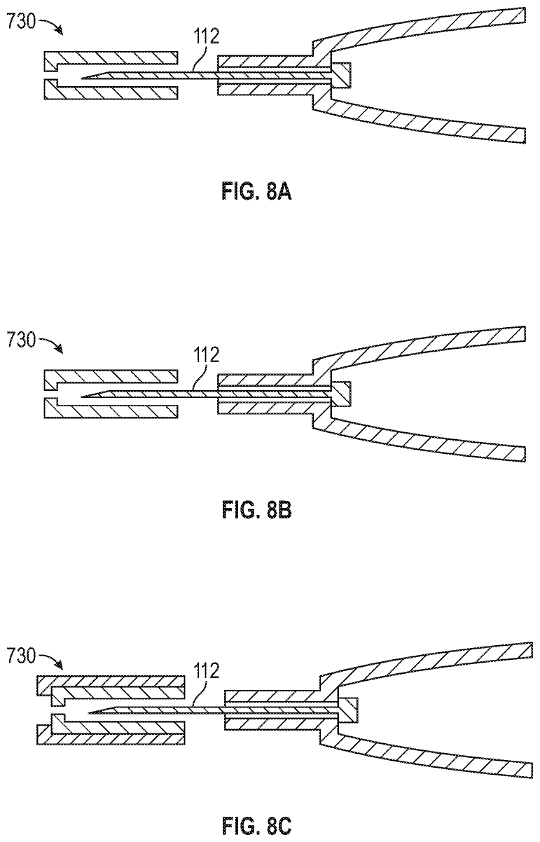

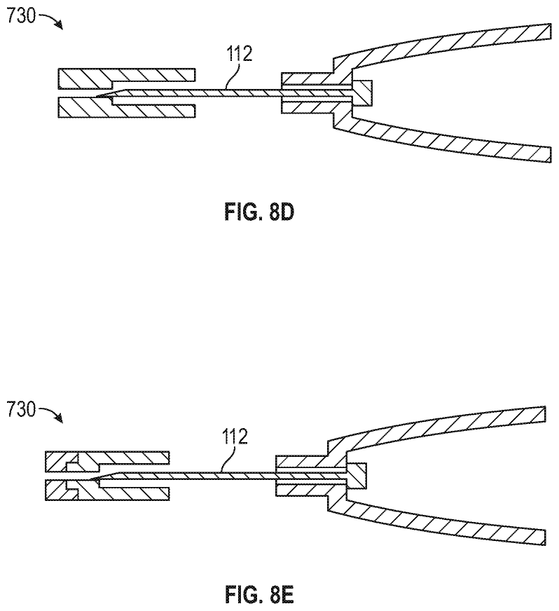

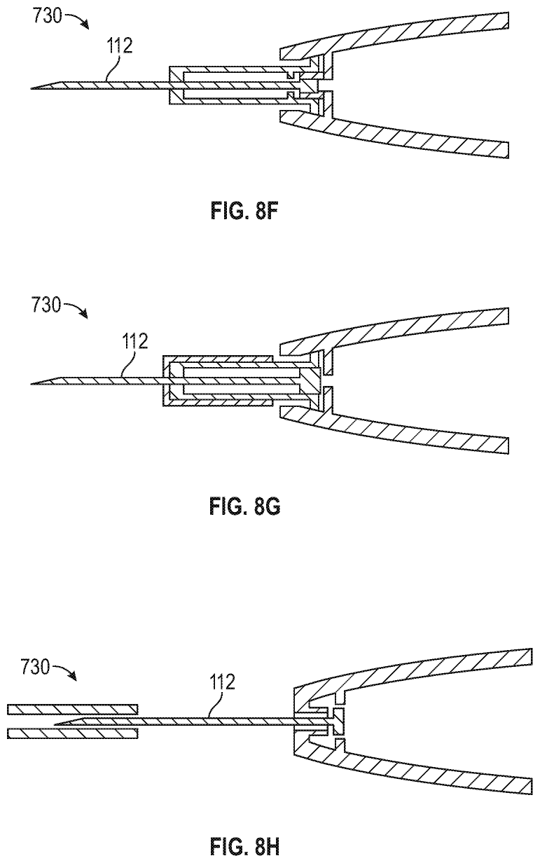

Referring to FIGS. 8A-8H, a needle stop 730 may be provided over needle 112 to understand the insertion depth of the needle and or prevent it from penetrating too deeply into the patient. For example, when the implant dispensing device is used to place the implant intravitreally, stop 730 may be configured such that its distal tip contacts the conjunctiva when the distal tip of needle 112 reaches the desired depth within the eye. Various alternative constructions for needle stop 730 are provided in FIGS. 8A-8H.

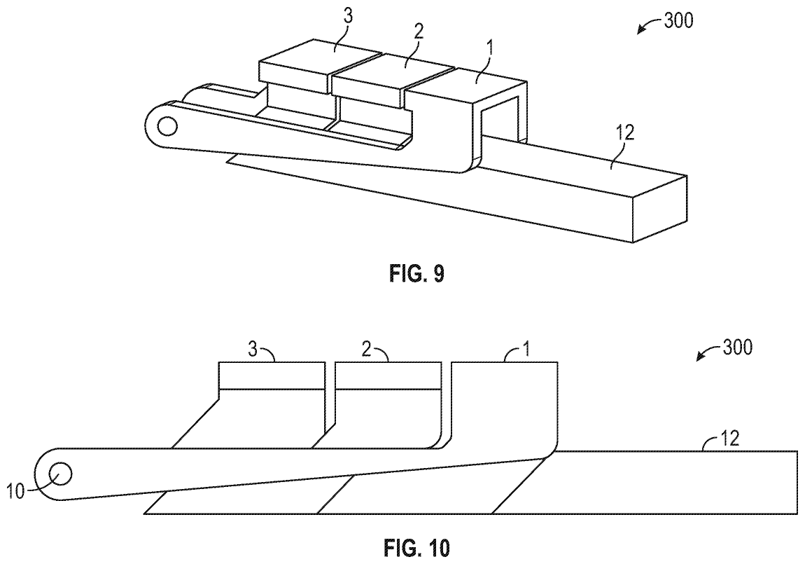

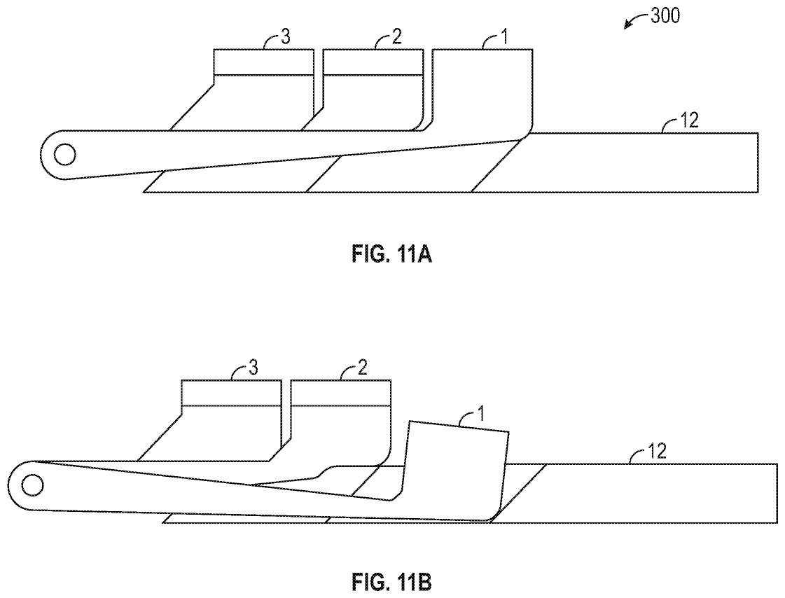

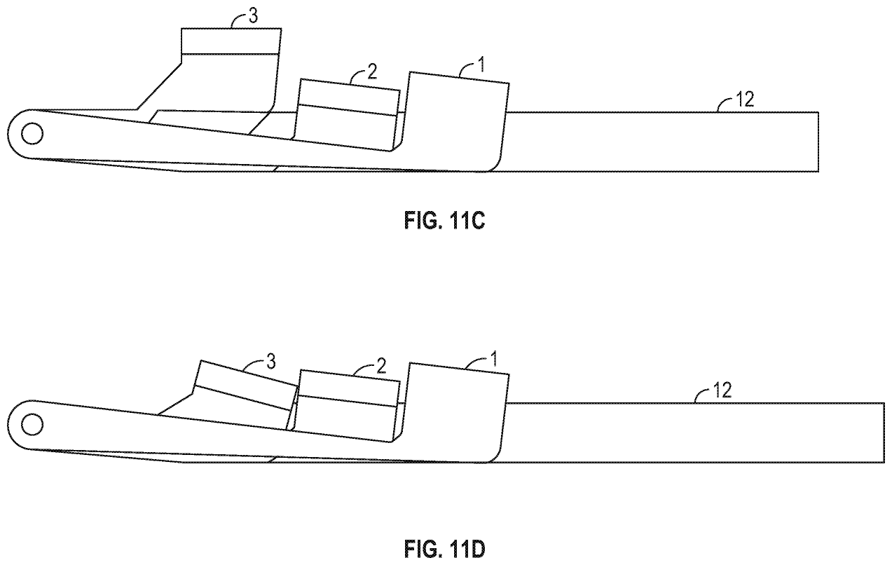

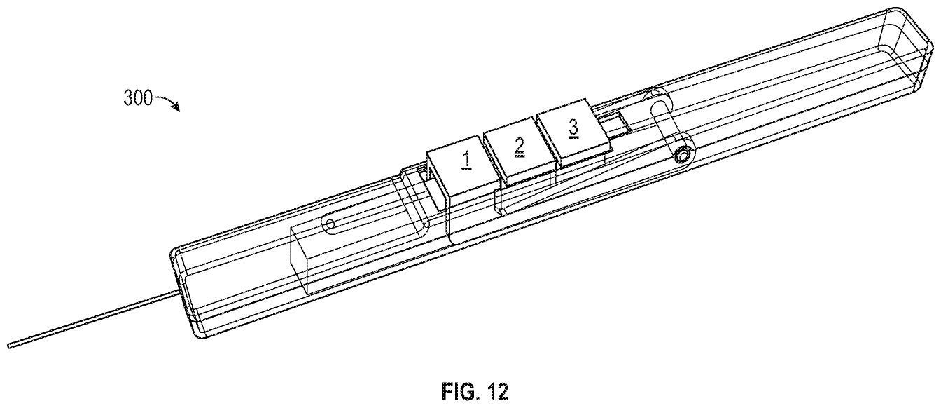

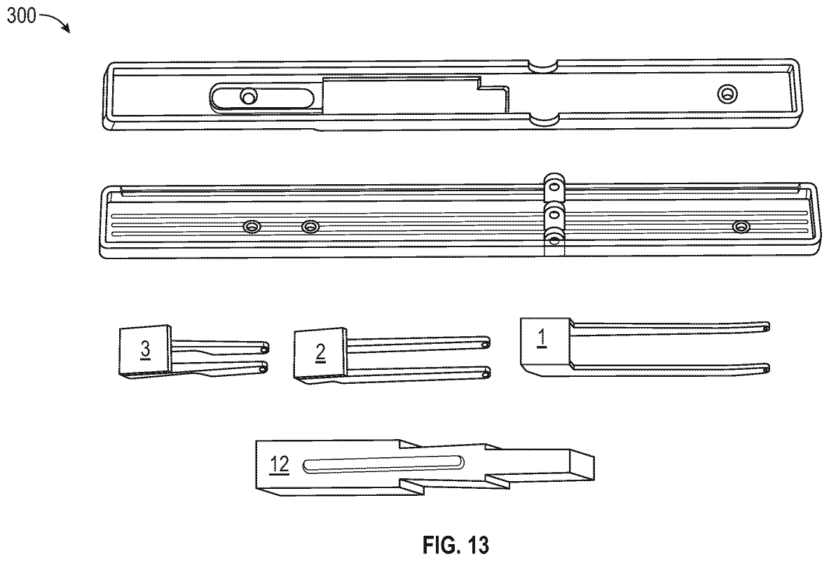

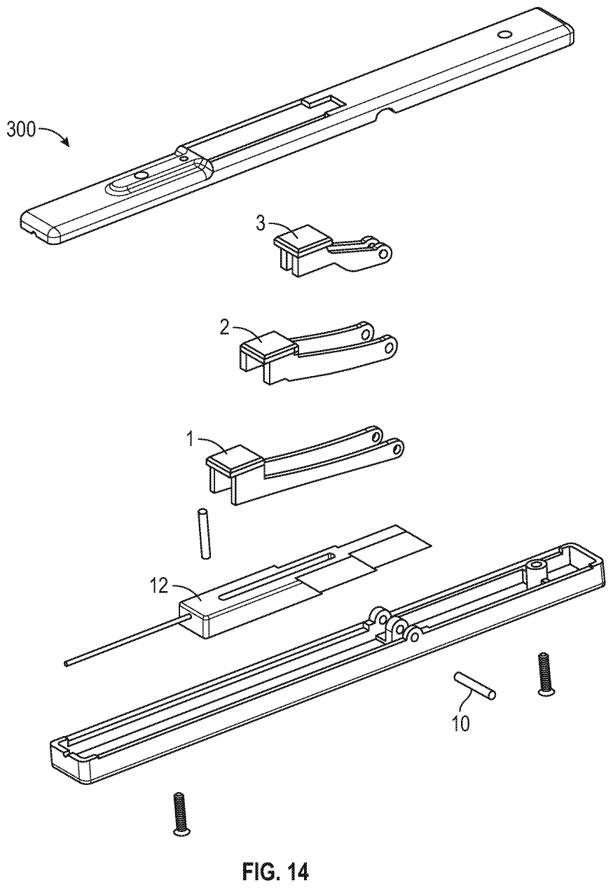

Referring to FIGS. 9-14, a second exemplary embodiment of an implant delivery device 300 is provided. Device 300 may be constructed and operated in a similar manner to device 100 previously described in reference to FIGS. 1-8. However, instead of dispensing a single implant, device 300 is configured to dispense multiple implants, such as three implants (also referred to as pellets.) Device 300 is provided with three dispense buttons, labeled 1, 2 and 3, one for each implant. All three buttons share a common pivot point around pin 10 (best seen in FIGS. 10 and 14.) Each button engages with a different ramped surface on a linear slide 12 to advance the slide further distally. Slide 12 is connected to a plunger 14 (shown in FIG. 14) which in turn pushes each implant sequentially out of a single needle when the associated button is depressed. FIGS. 11A-11D show and describe the sequence of dispensing the three implants/pellets by pushing buttons 1, 2 and 3 in successive order.

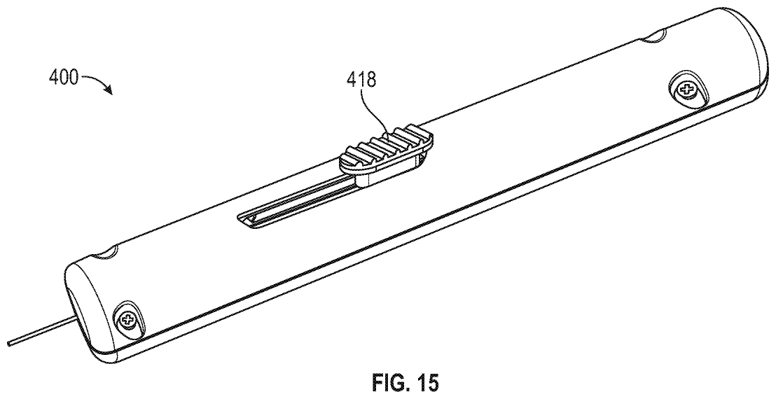

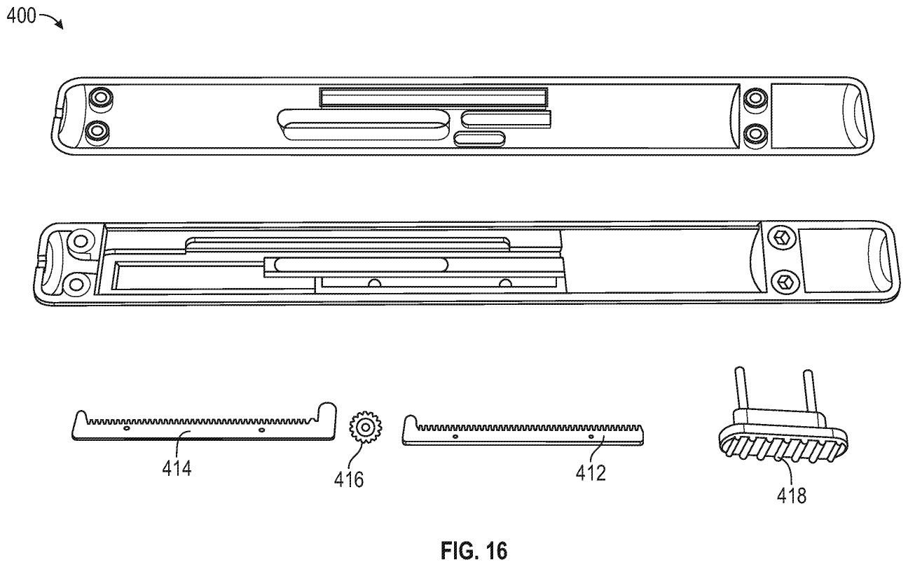

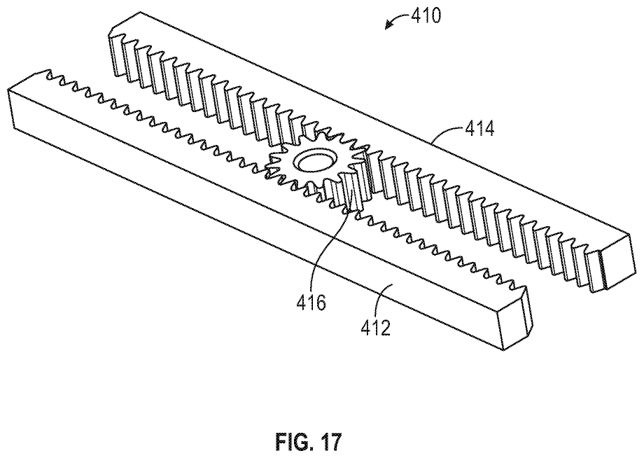

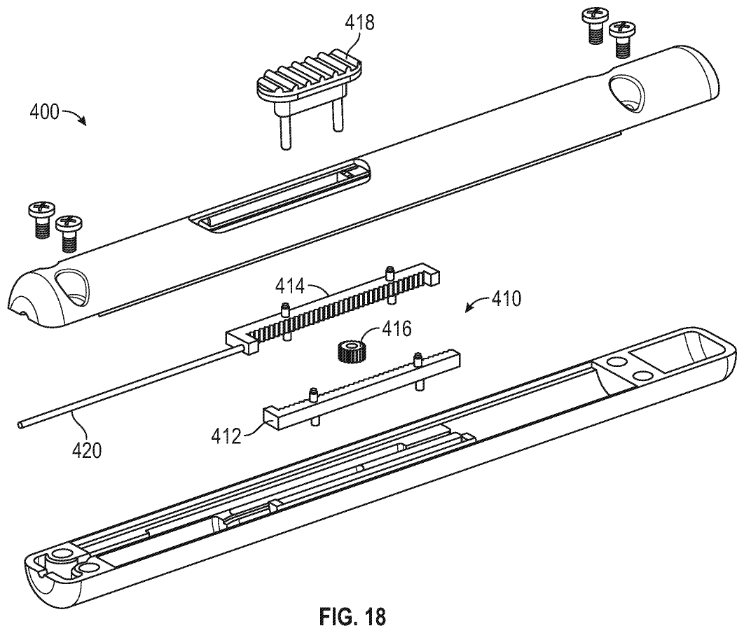

Referring to FIGS. 15-18, a third exemplary embodiment of an implant delivery device 400 is provided. Device 400 may be constructed and operated in a similar manner to device 100 previously described in reference to FIGS. 1-8. Device 400 may be configured to dispense a single implant or multiple implants, such as three implants (also referred to as pellets.) Device 400 may be provided with a rack and pinion mechanism 410, best seen in FIG. 17. Mechanism 410 includes a fixed rack 412, a floating rack 414 and a movable pinion 416 located between the two racks. Fixed rack 412 is affixed inside the device enclosure. Movable pinion 416 is attached to slide button 418. Floating rack 414 is slidably mounted inside the device enclosure and has a plunger 420 affixed to its distal end.

In operation, a medical practitioner slides button 418 distally from its proximal-most position. This moves pinion 416 an equal distance in the distal direction. As pinion 416 moves distally, it rotates in a counter-clockwise direction due to its gear-toothed engagement with fixed rack 412. This causes floating rack 414 and plunger 420 to also move distally but at twice the rate of the pinion. In other words, the distal movement of button 418 is magnified by rack and pinion mechanism 410. One or more detents may be provided on button 418 and or the enclosure so the medical practitioner can know when each of the one or more implants has been dispensed from device 400.

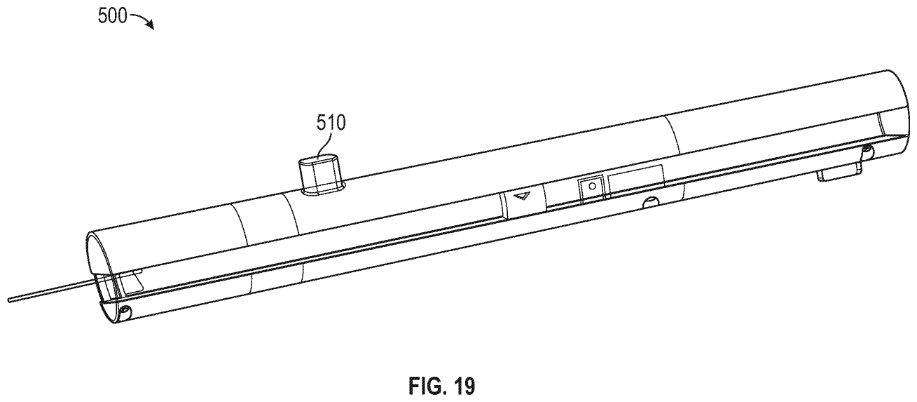

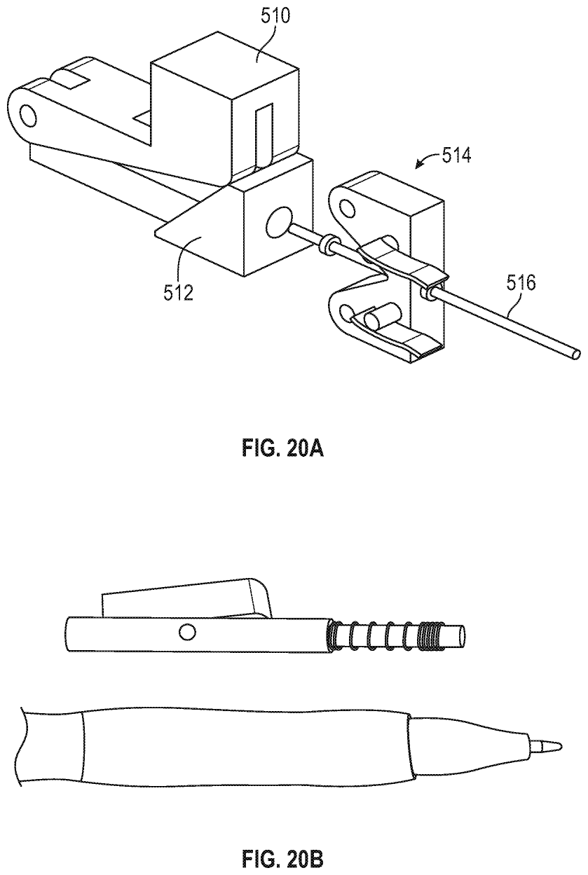

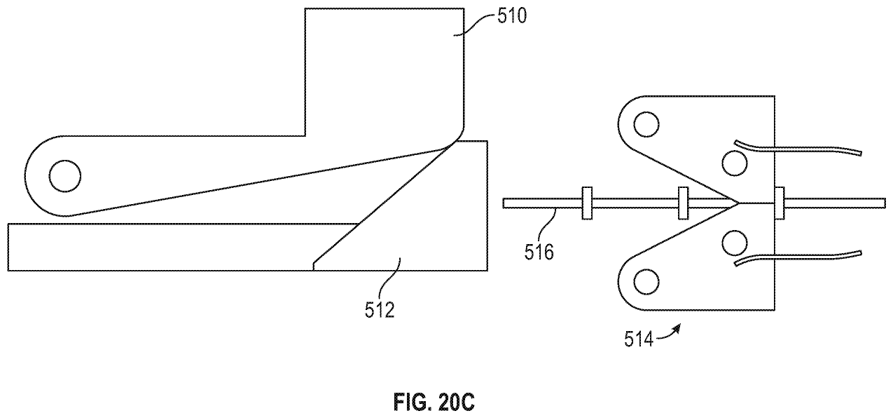

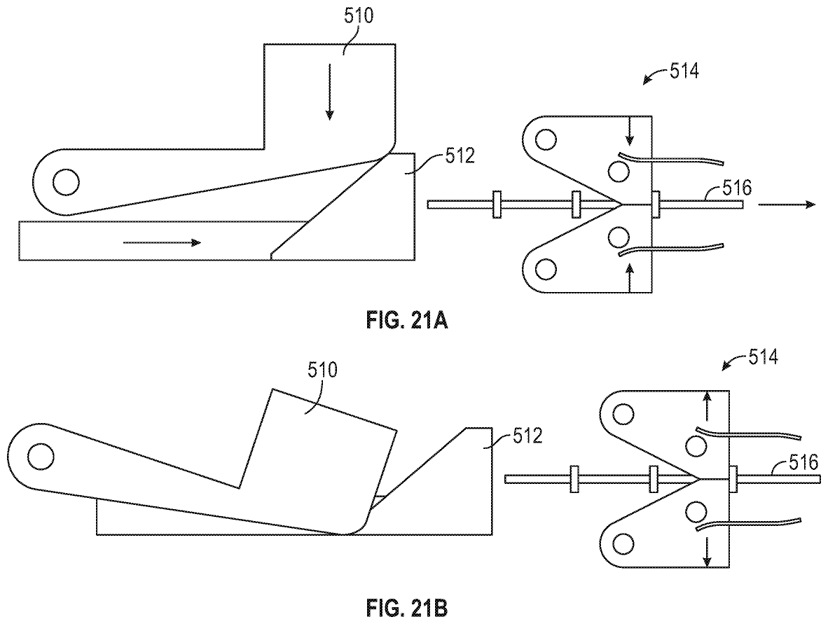

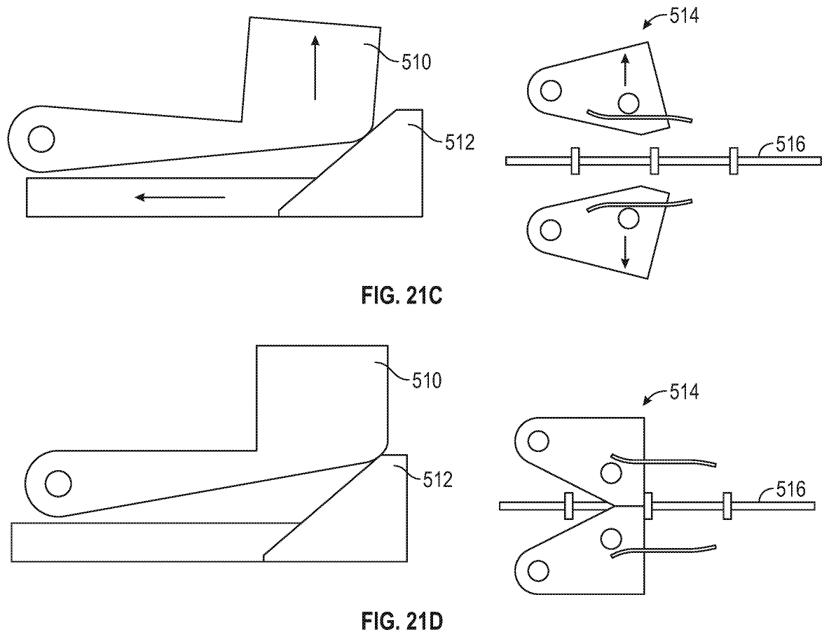

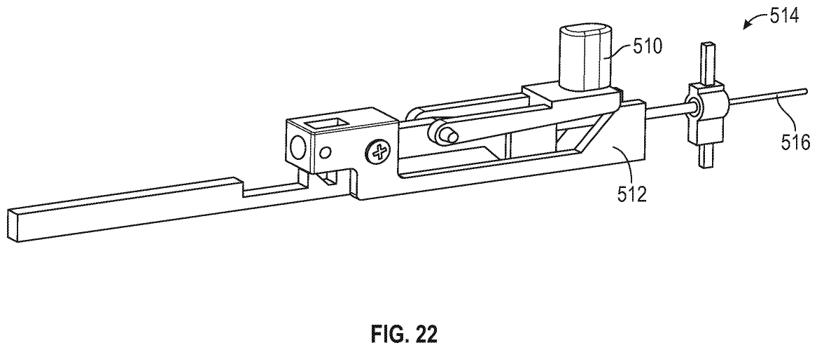

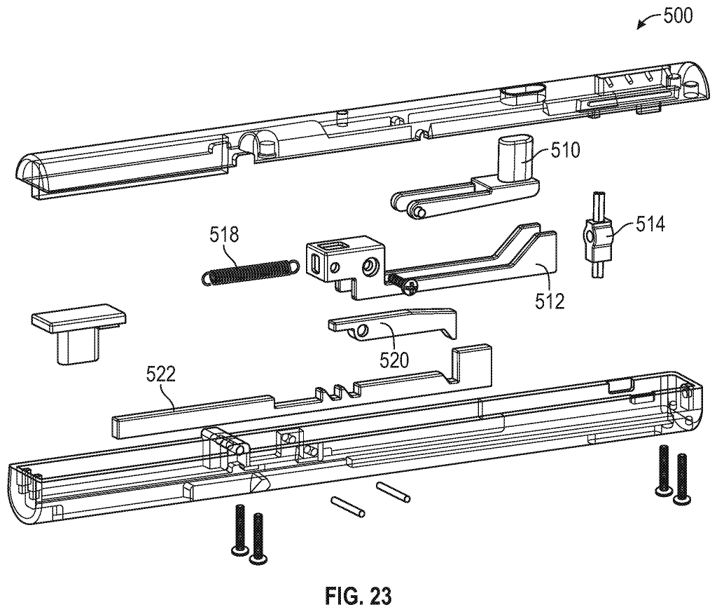

Referring to FIGS. 19-21, a fourth exemplary embodiment of an implant delivery device 500 is provided. Device 500 may be constructed and operated in a similar manner to device 100 previously described in reference to FIGS. 1-8. Device 500 may be configured to dispense a single implant or multiple implants, such as three implants (also referred to as pellets.) Device 500 employs a single button 510 that is pushed multiple times to dispense the one or more implants. As shown in FIG. 20A, device 500 utilizes a mechanism that includes a linear slide 512 that is driven towards the distal end of the device by button 510 each time it is pushed and then returns proximally when button 510 is released. A plunger grip jaw mechanism 514 allows plunger 516 to advance distally but not return proximally with linear slide 512. This arrangement is modeled on a mechanical pencil having a side push button, as shown in FIG. 20B. FIG. 20C is an enlarged cross-section of the components shown in FIG. 20A. FIGS. 21A-21D show and describe the reciprocating motion of the mechanism of FIG. 20A. FIG. 22 shows the mechanism of FIG. 20A specifically adapted for implant dispensing device 500, and FIG. 23 is an exploded perspective view that includes other components of device 500. As shown in FIG. 23, a return spring 518 may be coupled between slide 512 and the device enclosure to return slide 512 proximally after button 510 has been released. A pawl 520 may be pivotably mounted to slide 512 for driving rack 522 and the plunger in the distal direction. Each time slide 512 returns proximally, the distal end of pawl 520 progressively engages with a more proximally located notch on rack 522. The distal-most notch of rack 522 may be engaged by pawl 520 when device 500 is locked, and then move to the first of the three closely spaced notches when device 500 is primed. With the specific arrangement shown in FIG. 23, three implants may be successively dispensed from device 500. In other embodiments, the mechanism may be modified to dispense fewer or more than three implants.

While exemplary embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the invention and that methods and structures within the scope of these claims and their equivalents be covered thereby.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the disclosure. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and/or methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. When a feature is described as optional, that does not necessarily mean that other features not described as optional are required.

The examples and illustrations included herein show, by way of illustration and not of limitation, specific embodiments in which the subject matter may be practiced. As mentioned, other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implant delivery device comprising:
an elongated body having a proximal end and a distal end;
a cannula carried by the body and having a distal portion extending from the distal end of the body, the cannula being sized and configured to slidably receive a rod-shaped implant therein;
a plunger slidably received within the cannula and movable from a retracted position, in which a rod-shaped implant is retained inside the cannula, to a distally advanced position, in which the implant is ejected from the cannula by the plunger;
a dispense button movable from an extended position, in which the plunger remains in the retracted position, to a depressed position, in which the plunger is moved toward the distally advanced position; and
a prime button movable from a locked position, in which the dispense button is prevented from moving into the depressed position, to an unlocked position, in which the dispense button may be allowed to move toward the depressed position,
wherein the delivery device is configured to be operated by a user moving the prime button from the locked position to the unlocked position, and subsequently moving the dispense button from the extended position to the depressed position, thereby causing the plunger to move from the retracted position to the distally extended position to eject the implant from the distal portion of the cannula.

2. The device of claim 1, wherein the prime button is located on the proximal end of the elongated body and moves towards the distal end of the body when moving from the locked position to the unlocked position.

3. The device of claim 2, wherein the device is configured so that the prime button can be operated by a user's thumb.

4. The device of claim 3, wherein the dispense button is located on a top side surface of the elongated body and generally moves radially inward when moving from the extended position to the depressed position.

5. The device of claim 4, wherein the device is configured so that the dispense button can be operated by a user's forefinger or thumb.

6. The device of claim 1, wherein the device further comprises a cap movable from an attached position, in which the cap is covering the distal end of the body and the distal portion of the cannula, to a detached position, in which the cap is separated from the body.

7. The device of claim 6, wherein the device further comprises an interlock mechanism that prevents the cap from moving from the attached position to the detached position when the prime button is in the locked position.

8. The device of claim 6, wherein the device further comprises an interlock mechanism that prevents the dispense button from moving to the depressed position when the cap is in the attached position.

9. The device of claim 1, wherein the device further comprises a status window through a portion of the body, the status window allowing a user to see a position of a movable part within the body which represents a priming status and an implant status, the movable part being movable between a locked position, an unlocked position and a dispensed position.

10. The device of claim 9, wherein the position of the movable part is depicted by a change of color.

11. The device of claim 10, wherein the change of color comprises a red feature showing through the status window when the movable part is in the locked position, a yellow feature showing through the status window when the movable part is in the unlocked position, and a green feature showing through the status window when the movable part is in the dispensed position.

12. The device of claim 9, wherein the position of the movable part is depicted by a change of letter or symbol.

13. The device of claim 12, wherein the change of letter or symbol comprises an L showing through the status window when the movable part is in the locked position, a U showing through the status window when the movable part is in the unlocked position, and a D showing through the status window when the movable part is in the dispensed position.

14. The device of claim 1, wherein the device further comprises a spring configured to return the prime button to the locked position if the prime button is not fully moved to the unlocked position, and wherein the device is configured to provide tactile confirmation when the prime button is moved to the unlocked position, thereby confirming an unlocked state.

15. The device of claim 14, wherein the device is configured to emit a soft click when the prime button reaches the unlocked position.

16. The device of claim 1, wherein the device further comprises a spring configured to return the dispense button to the extended position if the dispense button is not fully moved to the depressed position, and wherein the device is configured to provide tactile confirmation when the dispense button is moved to the depressed position, thereby confirming an implant has been dispensed.

17. The device of claim 16, wherein the device is configured to emit a soft click when the dispense button reaches the depressed position.

18. The device of claim 1, wherein the cannula is provided with a retaining feature configured to releasably retain a rod-shaped implant within the cannula until the implant is pushed out by the plunger.

19. The device of claim 18, wherein the retaining feature comprises a bend in the cannula having an angle of at least 5 degrees.

20. The device of claim 18, wherein the retaining feature comprises one or more items in a group consisting of a bend, a nail head, a dimple, an oval, a flex section, an adhesive, an S-curve, an etched sleeve and a wire.

21. The device of claim 1, wherein the device further comprises a preloaded rod-shaped implant.

22. The device of claim 21, wherein the device further comprises a window through the elongated body allowing a user to view the preloaded implant before it is dispensed by the device.

23. The device of claim 1, wherein the device further comprises a push bar coupled to the plunger and configured to move longitudinally within the elongated body, the push bar configured to be moved distally by the prime button between a locked position and an unlocked position, the push bar configured to be moved further distally by the dispense button between the unlocked position and a dispensed position to drive the plunger distally within the cannula and dispense an implant from the distal portion of the cannula.

24. The device of claim 23, wherein the push bar is provided with cam features to unlock a cap positioned on the distal end of the body when the push bar reaches the unlocked position.

25. A device for delivering a rod-shaped implant, the device comprising:
an elongated body having a proximal end and a distal end;
a cannula carried by the body and having a distal portion extending from the distal end of the body, the cannula being sized and configured to slidably receive a rod-shaped implant therein;
a plunger slidably received within the cannula and movable from a retracted position, in which a rod-shaped implant is retained inside the cannula, to a distally advanced position, in which the implant is ejected from the cannula by the plunger;
a dispense button located on a top side surface of the elongated body and generally movable radially inward from an extended position, in which the plunger remains in the retracted position, to a depressed position, in which the plunger is moved toward the distally advanced position;
a cap movable from an attached position, in which the cap is covering the distal end of the body and the distal portion of the cannula and prevents the dispense button from moving into the depressed position, to a detached position, in which the cap is separated from the body and allows the dispense button to move into the depressed position;
a prime button located on the proximal end of the elongated body and movable towards the distal end of the body from a locked position, in which the cap is prevented from moving into the detached position, to an unlocked position, in which the cap is allowed to move into the detached position; and
a status window configured to indicate when the prime button has moved from the locked position to the unlocked position, and when the dispense button has moved into the dispense position,
wherein the delivery device is configured to be operated by a user moving the prime button from the locked position to the unlocked position, subsequently moving the cap from the attached position to the detached position, and subsequently moving the dispense button from the extended position to the depressed position, thereby causing the plunger to move from the retracted position to the distally extended position to eject the implant from the distal portion of the cannula.

26. A method of delivering a rod-shaped implant, the method comprising:
providing an implant delivery device including:
an elongated body with a proximal end and a distal end,
a cannula carried by the body and having a distal portion extending from the distal end of the body, the cannula carrying a rod-shaped implant therein,
a plunger slidably received within the cannula,
a dispense button located on the elongated body,
a cap removably attached to the distal end of the body and covering the distal portion of the cannula when the cap is attached to the body, and
a prime button located on the proximal end of the elongated body;
moving the prime button from a locked position to an unlocked position, thereby unlocking the cap;
after unlocking the cap by moving the prime button, removing the cap from the body, thereby unlocking the dispense button;
inserting a distal tip of the cannula into a patient; and
after unlocking the dispense button by removing the cap, moving the dispense button from an extended position to a depressed position, thereby causing the plunger to move distally to eject the implant from the distal portion of the cannula and into the patient.

27. The method of claim 26, wherein moving the prime button comprises placing a thumb on the button and moving the button in a distal direction.

28. The method of claim 26, wherein moving the dispense button comprises placing a forefinger on the button and moving the button in a radially inward direction.

29. The method of claim 26, wherein the rod-shaped implant is dispensed intravitreally.

30. The device of claim 1, wherein the device is configured to dispense the rod-shaped implant intravitreally.
